(19) United States
(12) Patent Application Publication
(10) Pub. No.: US 2006/0275248 A1
(43) Pub. Date: Dec. 7, 2006

(54) METHODS AND DEVICES FOR INFANT FEEDING

(75) Inventor: Robert Alexander OSTERBAUER, Neu-Anspach (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: EPISODE 1 PARTNERS LTD, London (GB)

(21) Appl. No.: 11/421,836

(22) Filed: Jun. 2, 2006

(30) Foreign Application Priority Data
Jun. 2, 2005 (GB) 0511260.2

(51) Int. Cl. A61K 9/70; A61L 9/00

(52) U.S. Cl. 424/76.2; 424/443

(57) ABSTRACT

A method of feeding an infant, the method comprising the steps of: providing a feeding device; adding a female's breast odor to the feeding device; and feeding the infant using the feeding device. A feeding device, a pacifying device and an odor carrier are also described.

### BACKGROUND OF THE INVENTION

This invention relates to infant care. In particular, the invention relates to a method of feeding infants, a feeding device, a method of pacifying infants, a pacifying device and an odor carrier.

Many infants are currently not breastfed but instead are fed with a feeding device, for example a baby bottle containing formula or expressed milk. The reasons for this are manifold: mothers may not produce sufficient milk to satisfy the nutritional needs of their infant, mother and infant might be separated for medical reasons (e.g. in the case of a premature birth), a mother may prefer to use a breast pump to extract breast milk, or a mother may choose not to breast feed for cosmetic or social reasons.

Feeding infants with a feeding device, although necessary and/or desirable in many situations, can be fraught with difficulties. It is common for infants to refuse nutrition from a feeding device, particularly if they were previously breast fed or if they are subjected to a new diet. This can lead to a reduction in nutrition and fluid intake by the infant, which is generally harmful.

A further problem with feeding infants with a device is the resultant diminished mother-child bonding when compared to breast feeding. Infants often become unhappy and restless when they sense that their mother is not nearby, particularly if they are hungry. Furthermore, in the context of feeding, although diminished bonding is particularly problematic when a baby's mother is not present at all, even infants fed with feeding devices by their own mother partly miss out on the feeling of closeness and bonding offered by breast feeding. It is thought that close contact between mothers and infants has a strong formative effect on their life long relationships with each other.

There have been numerous attempts to mitigate the problem of nutrition refusal. Improved nutrition formulas have for instance been suggested. Furthermore, feeding devices themselves have been adapted to allow nutrition to be taken in more easily and to make infants feel at ease. French Patent Publication FR2595569, for example, describes a multi-sensory baby bottle made of rubber that in its shape, texture and flexibility imitates the anatomy of a female breast and includes an adhesive collar impregnated with a mother's smell, thereby providing a bottle that is said to reassure and improve the comfort levels of infants during feeding.

Unfortunately known feeding devices that mimic a mother's presence, even when combined with improved nutrition formulations do not overcome the problems of nutrition refusal and diminished bonding. The factors that contribute to an infant's relative happiness when it is breastfed and/or in the presence of its mother are numerous, of varying importance, but not well understood in the prior art. As a result, existing measures to enhance nutritional intake from feeding devices, or simply mimic the presence of a mother, are not effective.

### BRIEF SUMMARY OF THE INVENTION

It is an object of the present invention to provide a method and device that mitigates at least some of the problems associated with the prior art. In particular, the present invention aims to provide a method and device facilitating at least one of the following: mother-infant bonding; transition from breastfeeding to bottle feeding; acceptance of novel food products by infants; promotion of sucking behaviour in infants; increased fluid intake by infants; and pacifying infants where feeding is not appropriate.

From a first aspect, the invention broadly resides in a method of feeding an infant, the method comprising the steps of: providing a feeding device; adding a female's breast odor to the feeding device; and feeding the infant using the feeding device.

The term “odor” wherever used throughout this specification refers to any quality that may be perceived by the olfactory sense, be it consciously (e.g. pleasant smells) or sub-consciously (e.g. pheromones). An odor may comprise a single component or a mixture of components.

One reason the method according to the first aspect of the invention represents an improvement over the prior art is that it provides, in the presence of a feeding device, the essential olfactory cues that an infant would normally experience during breastfeeding. The specific olfactory cues from a female's breast are vital in improving mother-infant interaction and facilitate attachment and bonding. Furthermore, the chemical signals from a female's breast region contain putative pheromones that instigate suckling behaviour, help in guiding infants' movements, and therefore increase nutrition intake.

The first aspect of the invention is particularly suitable for weaning infants because it provides, during artificial feeding, natural olfactory cues expected by an infant. This means that feeding devices are less likely to be refused because they are unfamiliar, even if the diet of the infant is changed, for example from breast milk to an artificial formula.

Both artificially and naturally produced female breast odor may be employed according to the first aspect of the present invention. However, since naturally produced odors are freely available, the use of these is preferred.

The applicant has found that it is beneficial for the female's breast odor to be that of a lactating female. Infants recognise odor originating from a lactating female's breast and, in the context of feeding, prefer it, for example, to other body odors of a lactating female and the breast odor of non-lactating females.

Ideally, the female's breast odor is that of the infant's mother; breastfed infants in particular become familiar with, and respond preferentially to, their mother's odor. The mother's odor may preferably be that of the infant's biological mother although the odor of an adopted mother may also be used.

As a particularly convenient way of adding a female's breast odor to the feeding device, the method according to the first aspect of the invention may comprise the sub-steps of: providing an odor carrier; bringing the odor carrier into contact with a female's breast to allow the breast odor to be absorbed; and combining the odor carrier with the feeding device. Optionally, prior to bringing the odor carrier into contact with the female's breast, and as a further sub-step, the method may include treating the odor carrier with a solvent, emollient, biological additive, other substance, or combination thereof, for example to improve the odor carrier's odor retaining ability. Substances applied to the odor carrier can not only perform the function of allowing greater odor absorption but may also, additionally or alternatively, provide a skin treatment, for example to prevent irritation. Glyceryl esters in general and propylene glycol in particular represent examples of substances that may preferably be applied to the odor carrier.

Conveniently, the odor carrier may be held in contact with the female breast by being attached thereto, for example with an adhesive. Alternatively or additionally, the odor carrier can be held in contact with the female breast by being attached to an article of clothing, again for example with an adhesive. Contact over a period of several hours is preferred for good levels of odor absorption.

Where an odor carrier is brought into contact with a breast, it is envisaged that the method may further comprise the sub-step of using the odor carrier as a nursing pad for absorbing leaked breast milk, prior to combining the odor carrier with the feeding device.

Using the odor carrier as a nursing pad prior to combining it with a feeding device is a convenient and a highly effective way of adding a female's breast odor to the feeding device and simultaneously allows it to perform a useful secondary function.

Since it may, for example, not be convenient to combine the entire odor carrier with the feeding device, a section of the odor carrier may be discarded, after the odor carrier is used as a nursing pad but prior to combining the odor carrier with the feeding device.

Furthermore, in cases where the odor carrier is used as a nursing pad for lactating females who may be prone to leak milk, the discarded section of the odor carrier may be selected on the basis of the distribution of leaked breast milk across the odor carrier. Ideally, the discarded section of the odor carrier is selected to contain a higher concentration of leaked breast milk than the remainder of the odor carrier. Eliminating those parts of the odor carrier with a higher concentration of leaked breast milk may be desirable since breast milk is relatively unstable and can produce malodorous by-products, which may have an adverse effect on the working of the present invention.

From a second aspect, the present invention broadly resides in a feeding device for feeding an infant, the device including nutrition storage means and a nutrition dispenser for dispensing stored nutrition, wherein the device is adapted to co-operate with an odor carrier carrying a female's breast odor such that the infant can be exposed to the female breast odor during feeding.

Preferably, the feeding device further comprises an odor carrier carrying a female's breast odor for exposing the infant to the female breast odor during feeding.

Both artificially and naturally produced female breast odors may be carried by the odor carrier. However, since naturally produced odors are freely available, the use of these is preferred.

The applicant has found that it is beneficial for the female's breast odor to be that of a lactating female. Infants recognise odor originating from a lactating female's breast and, in the context of feeding, prefer it, for example, to other body odors of a lactating female and the breast odor of non-lactating females.

Ideally, the female's breast odor is that of the infant's mother; breastfed infants in particular become familiar with, and respond preferentially to, their mother's odor. The mother's odor may preferably be that of the infant's biological mother although the odor of an adopted mother may also be used.

It should be noted that although the feeding device according to the second aspect of the invention is particularly suitable for exposing an infant to a female's breast odor during feeding, it may also carry other odors as required. It is possible to use the feeding device according to the second aspect of the invention to provide feeding infants with the olfactory cues of other human natural odors such as male or female axillary or neck odor, artificially produced human odors, food odors or perfume. It is also possible to assemble the feeding device for use without the odor carrier.

The odor carrier may be formed of any material suitable for carrying odor, such as, for example cotton, cellulose or a fabric. It may be treated with a solvent, emollient, biological additive, other substance, or combination thereof, for example to improve its odor retaining ability. Substances applied to the odor carrier can not only perform the function of allowing greater odor absorption but may also, additionally or alternatively, provide a skin treatment, for example to prevent irritation. Glyceryl esters in general and propylene glycol in particular represent examples of substances that may preferably be applied to the odor carrier.

To infuse the odor carrier with odor, it may be held in contact with an appropriate region of skin until it has absorbed a sufficient amount of odor before being used in the feeding device. Thus, for example, if odor from the breast region is desired the odor carrier can be fastened to a breast or a bra using an adhesive. Alternatively or additionally, if neck odor is desired, the odor carrier can be held in place in the neck region by fastening it to clothing and/or appropriate skin. In the case of artificial odors it may be more convenient simply to apply a few drops of an appropriate liquid directly to the odor carrier.

To enhance the amount of odor the infant is exposed to, the odor carrier may be located in the vicinity of the feeding device's nutrition dispenser. The nutrition dispenser may advantageously comprise a teat and the odor carrier may be adapted to co-operate with said teat.

The odor carrier may beneficially be housed below at least one outer surface of the feeding device. This minimises contamination of the odor carrier with nutritional or other odors it may otherwise come into contact with.

Where the odor carrier is housed below at least one outer surface, the or each surface may comprise at least one diffusion opening for allowing odor to diffuse from the odor carrier and out of the feeding device. Shutter or cover means may be present to allow at least one diffusion opening to be opened and closed. The diffusion openings may be of any size suitable for allowing odor to diffuse. In particular, they may have an average cross section of 1 mm or be pore sized.

From a third aspect, the present invention broadly resides in a pacifying device for pacifying an infant, characterised in that it is adapted to co-operate with an odor carrier carrying a female's odor such that the infant can be exposed to the female's odor when using the pacifier.

The pacifying device preferably comprises an odor carrier carrying a female's odor. Both artificially and naturally produced female breast odor may be carried by the odor carrier. However, since naturally produced odors are freely available, the use of these is preferred.

Since the pacifying device may provide an infant with a female's olfactory cues with the help of the odor carrier, it can increase comfort levels without the need for a female to be in constant close proximity.

Ideally, the female odor may be that of the infant's own mother, preferably it's biological mother. Incorporating the smell of the infants own mother has the added advantage of improving bonding between infant and mother whenever the pacifying device is used.

The odor carrier may be formed of any material suitable for carrying odor, such as, for example, cotton, cellulose or a fabric. It may be treated with a solvent, emollient, biological additive, other substance, or combination thereof, for example to improve its odor retaining ability. Substances applied to the odor carrier can not only perform the function of allowing greater odor absorption but may also, additionally or alternatively, provide a skin treatment, for example to prevent irritation. Glyceryl esters in general and propylene glycol in particular represent examples of substances that may preferably be applied to the odor carrier.

It is preferred that the female odor carried by the odor carrier is that of the infant's own mother. Infants prefer their mother's odor to the odor of other females. Ideally, the mother's odor may be that of the infant's biological mother although the odor of an adopted mother may also be used.

Infusion of the odor carrier may be carried out as described in respect of the odor carrier that may form part of the feeding device according to the second aspect of the invention.

It should be noted that odors sampled from the nipple region may not have the desired effect when used in a pacifying device. Presenting an infant with feeding-related odor cues in the absence of food can lead to agitation. The invention as seen from the third aspect envisages the use of a broad variety of female odors including breast odor, but preferably utilises axillary odor and/or neck odor. It is also possible to use male odors or entirely artificial odors such as food odors, perfumes or pheromone mixtures.

It is preferred that the pacifying device comprises a teat and that the odor carrier is adapted to co-operate with said teat.

Advantageously, the odor carrier may be housed below at least one outer surface of the pacifying device. This helps to minimise contamination of the odor carrier in use.

Where the odor carrier is housed below at least one outer surface, the or each surface may comprise at least one diffusion opening for allowing odor to diffuse from the odor carrier and out of the pacifying device. Shutter or cover means may be present to allow at least one diffusion opening to be opened and closed. The diffusion openings may be of any size suitable for allowing odor to diffuse. In particular, they may have an average cross section of 1 mm or be pore sized.

From a fourth aspect the present invention broadly resides in a method of pacifying an infant, the method comprising the steps of providing a pacifier, adding a female odor to the pacifier and pacifying the infant using the pacifier.

The various preferred features described in relation to the pacifier according to the third aspect of the invention give rise to equivalent preferred features of the method according to the fourth aspect of the invention.

From a fifth aspect, the present invention broadly resides in an odor carrier comprising an absorbent portion for absorbing odor, and being adapted to co-operate with a feeding or pacifying device such that an infant is exposed to odor absorbed by the odor carrier.

The odor carrier may preferably be adapted to be usable as a nursing pad, thereby allowing it to perform a double function. It may be formed of any material suitable for carrying odor, such as, for example cotton, cellulose or a fabric.

Conveniently, the odor carrier may be treated with a solvent, emollient, biological additive, other substance, or combination thereof, for example to improve its odor retaining ability. Substances applied to the odor carrier can not only perform the function of allowing greater odor absorption but may also, additionally or alternatively, provide a skin treatment, for example to prevent irritation. Glyceryl esters in general and propylene glycol in particular represent examples of substances that may preferably be applied to the odor carrier.

Infusion of the odor carrier (with any odor) may for example be carried out as described in respect of the odor carrier that may form part of the feeding device according to the second aspect of the invention. Once infused, an odor carrier according to the fourth aspect of the invention may for example be employed in a feeding device according to the second aspect of the invention or a pacifying device according to the third aspect of the invention.

In the event that an odor carrier needs to be stored prior to being used it may be placed into an airtight container, preferably at low temperature (e.g. −20° C.). This serves to reduce the amount of odor dispersed from the pad prior to use. Storage of the odor carrier is particularly important since the odor carrier disperses odor in use and therefore needs to be periodically replaced or re-infused with odor to maintain its effectiveness.

Conveniently, the odor carrier comprises a pad in the shape of a disk, which may or may not be annular and may optionally be made of cotton.

Advantageously, the pad may comprise a removable section, which may be disk shaped and concentric with the disk shaped pad. The removable section may lie in the same plane as the remainder of the odor carrier, or in a different plane.

Preferably, the removable section may be separated from the remainder of the disk shaped pad by a perforated border.

The pad may comprise an indent for accommodating the nipple of a breast. Where the odor carrier comprises a removable section, the indent may beneficially be located on the removable section.

It is also a preferred feature that the odor carrier comprises a liquid impermeable barrier for preventing the spread of liquid to a part of the pad. This may, for example, be advantageous in the context of preventing the spread of leaked breast milk. Where the odor carrier comprises a removable section, the barrier may be located in the removable section and be suitable for preventing the spread of liquid out of the removable section; in an alternative arrangement, the barrier may be located between the removable section and the remainder of the pad.

Alternatively, or in addition to the relevant preferred features already described, the pad may comprise at least one slit to allow the odor carrier to co-operate with a feeding or pacifying device.

The odor carrier may comprise an adhesive portion for affixing the odor carrier to human skin. Alternatively or in addition, the odor carrier may comprise an adhesive portion for affixing the odor carrier to an item of clothing. The adhesive portions may each be separate or integral with the absorbent portion.

The presence of an adhesive portion, or the application of adhesive to one side of the odor carrier enhances the usability of the odor carrier as a nursing pad. It is important that the odor carrier is held in place to absorb odor and/or breast milk efficiently and adhesive is a convenient way of doing so.

It is beneficial for the odor carrier to be adapted to be held below at least one outer surface of a feeding or pacifying device to prevent contamination.

It will be appreciated that the present invention serves to expose infants to an appropriate odor in at least two situations, namely feeding and pacifying, providing comfort and reassurance in a manner not previously contemplated.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 depicts the main components of a first embodiment of the invention;

FIG. 2 is a side view of the embodiment of FIG. 1 in an assembled state, with uncovered diffusion openings;

FIG. 3 depicts the main components of a second embodiment of the invention;

FIG. 4 is a perspective view of the embodiment of FIG. 3 in an assembled state, with uncovered diffusion openings;

FIG. 5 depicts a first alternative odor carrier;

FIG. 6 depicts a second alternative odor carrier;

FIG. 7 depicts a third alternative odor carrier;

FIG. 8 depicts a fourth alternative odor carrier; and

FIG. 9 depicts a fifth alternative odor carrier.

### DETAILED DESCRIPTION OF THE INVENTION

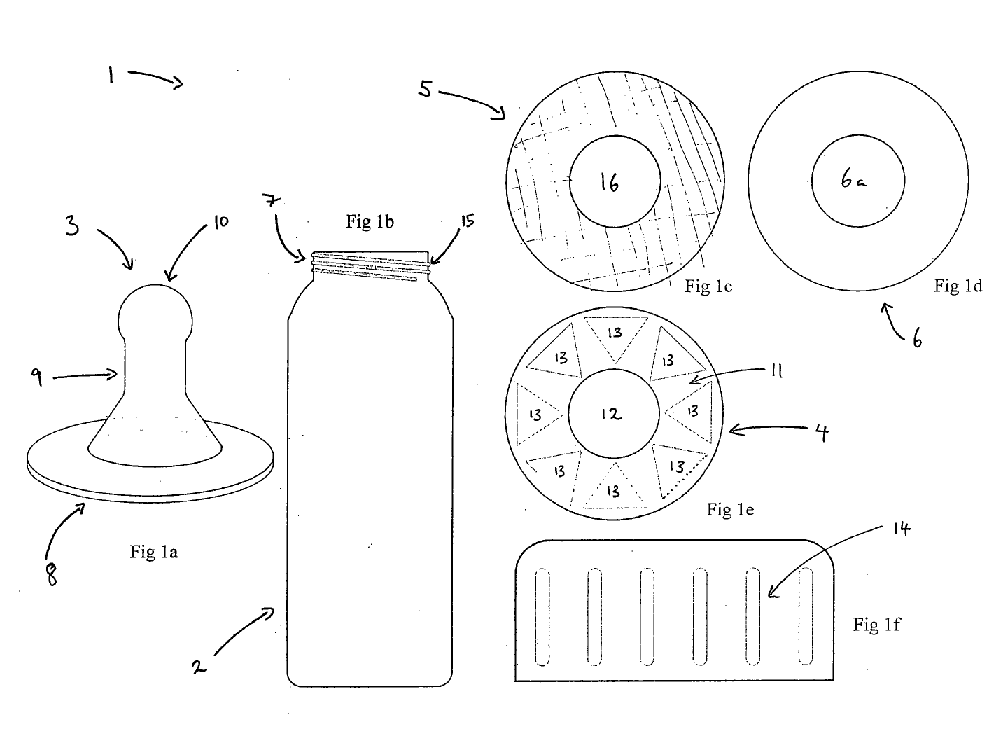

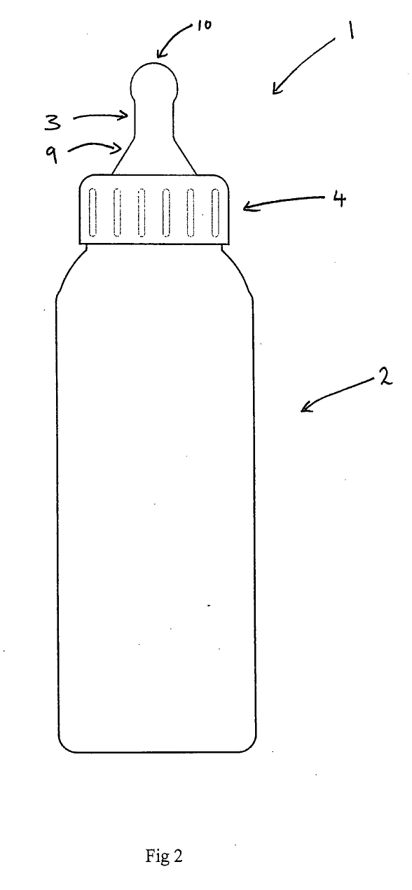

Referring firstly to FIGS. 1 and 2, in a first embodiment of the invention a feeding device 1 comprises nutrition storage means in the form of a plastic container 2, a nutrition dispenser in the form a teat assembly 3, a fastening mechanism in the form of a container cap 4, an odor carrier in the form of an annular absorbent pad 5 made of cotton, and a shutter (cover) 6 in the form of an annular rubber ring.

The plastic container 2 has an upper end 7 comprising an opening for receiving nutrition. In use, once the container 2 has been filled with nutrition, its opening is covered by the teat assembly 3, which is held in place by the container cap 4. The absorbent pad 5 is infused with a mother's breast odor and then held in place between the teat assembly 3 and the cap 4 to provide an infant with olfactory cues when ingesting nutrition from the container via the teat assembly 3. The cover 6 is placed on top of the container cap 4 when the feeding device 1 is not in use.

To allow the teat assembly 3 to perform its function of covering the opening of the plastic container 2, it comprises a base portion 8 in the shape of an annular disc having an outer diameter marginally greater than that of the opening in the plastic container 2. The base portion 8 of the teat assembly 3 is thus able to rest on the rim of the opening of the plastic container 2, but without significant overlap. A second key component of the teat assembly 3 is the teat 9 itself, which is formed conventionally, from a flexible latex or silicone layer and extends from the rim of a central circular opening in the annular base portion 8, tapering to form a bulbous end 10 comprising an opening and suitable for being suckled by an infant.

It will be appreciated that in order for the teat assembly 3 effectively to function as a nutrition dispenser via the bulbous end 10 of the teat 9, it must be sealed to the opening of the plastic container 2 at its base portion 8. This is achieved by means of the container cap 4, which simultaneously performs the function of holding the annular absorbent pad 5 in place in the vicinity of the teat assembly 3, as described below.

The container cap 4 comprises a planar portion 11 in the shape of an annular disc having upwards and downwards facing flat surfaces and an outer diameter and central opening 12 similar in dimension to those of the base portion 8 of the teat assembly 3. A plurality of diffusion openings 13 are formed in the planar portion 11 and radially surround the central circular hole 12. The annular cover 6 comprising a central opening 6a is placed removably on the upwards facing surface to enable the diffusion openings 13 to be closed when desired, for example if the device 1 is not in use. Extending perpendicularly downwards from the outer perimeter of the planar portion 11 is an annular edge portion 14, which comprises on its inward facing surface a first screw thread (not shown) suitable for engaging with a second complementary screw thread 15 formed on the outer surface of the plastic container 2, in the vicinity of the plastic container's opening.

To seal the teat assembly 3 to the plastic container 2 and simultaneously hold in place the annular absorbent pad 5, the teat assembly 3 is first placed on the rim of the plastic container's opening, with the teat 9 extending away from the plastic container 2. The annular absorbent pad 5, also having a diameter and a central opening 16 of similar dimensions to the base portion 8 of the teat assembly 3, is then placed horizontally on top of the base portion 8 of the teat assembly 3 to surround the teat 9 at its base. Finally, the container cap 4 is lowered onto the annular absorbent pad 5, with its annular edge portion 14 facing towards the plastic container 2 and the teat 9 of the teat assembly 3 projecting through the central opening 12 of the planar portion 11. The cap 4 is secured in place firmly by screwing it to the upper end 7 of the plastic container 2 by means of the complementary screw threads.

The feeding device 1 according to the first embodiment of the invention represents a modification of a known feeding bottle. The arrangement described above is similar to those seen in conventional feeding bottles, but differs as a result of the inclusion of the annular absorbent pad 5 the diffusion openings 13 in the container cap 4 and the cover 6. It is a feature of the present invention to add a female's breast odor to a feeding device and the modifications introduced in the first embodiment described above specifically facilitate this.

Prior to being combined with the remaining components of the feeding device 1 according to the first embodiment, the annular absorbent pad 5 is held in contact with a female's breast (not shown) to absorb odors emanating from the skin. This can be achieved by fastening the pad 5 to the breast itself and/or the inside of a bra (not shown), for example with adhesive. Once the annular absorbent pad 5 is held in place between the container cap 4 and the teat assembly 3, it disperses absorbed odors via opened diffusion openings 13 in the container cap 4, providing olfactory cues to an infant (not shown) feeding from the feeding device 1. When the device 1 is not in use the diffusion openings 13 are closed by the cover 6 to reduce the dispersion of odor from the odor carrier 5.

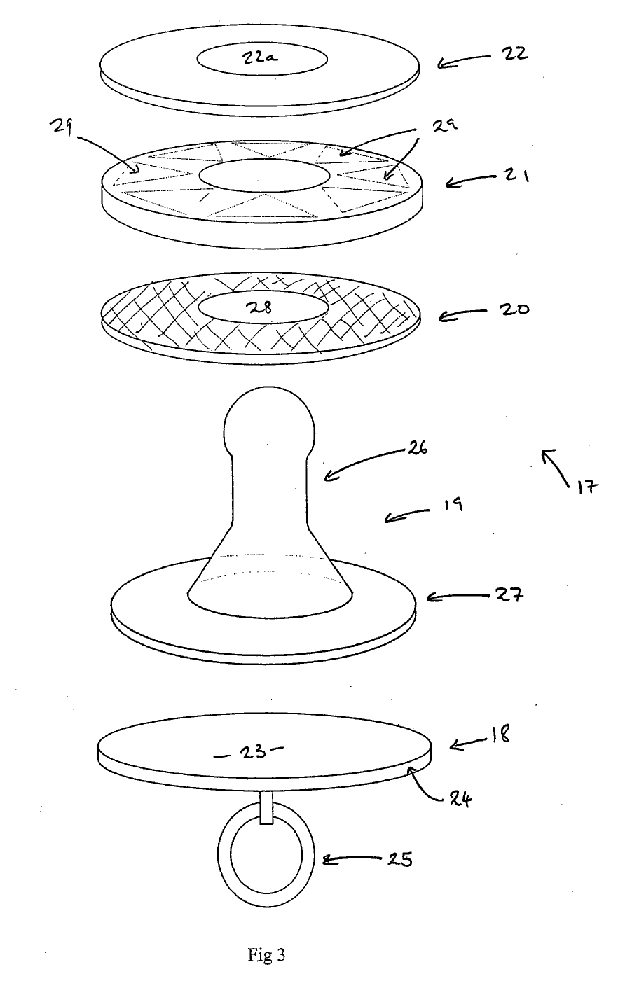

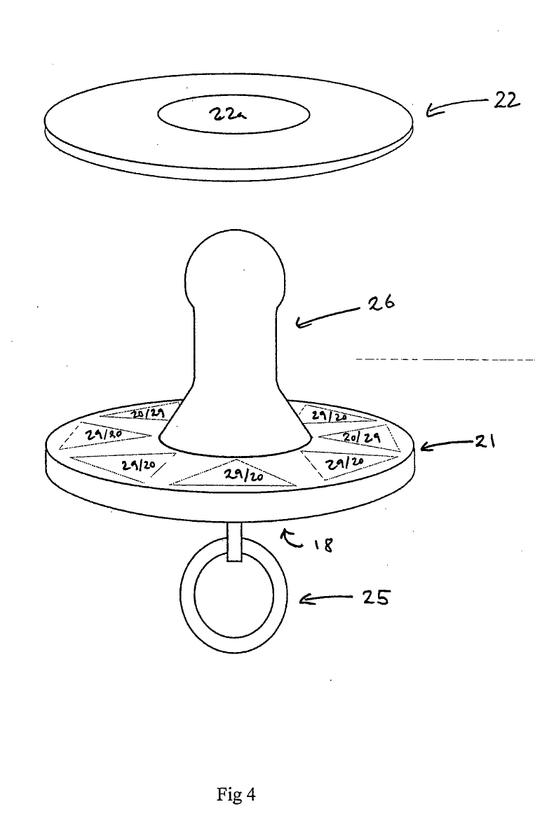

Referring now to FIGS. 3 and 4, in a second embodiment of the invention a pacifier 17 comprises a backing section 18, a teat assembly 19, an odor carrier in the form of an annular absorbent pad 20 made of cotton, a cap 21 and a shutter (cover) 22 in the form of an annular rubber ring.

The backing section 18 comprises a disc having upper planar surface 23, a lower planar surface (not shown), and a rim surface 24 having a first screw thread (not shown) thereon. A ring shaped handle 25 is attached to the lower planar surface of the backing section 18, whilst the upper planar surface 23 provides support for the teat assembly 19.

The teat assembly 19 of the second embodiment has the same structure as the teat assembly 3 of the first embodiment, with the exception that the teat 26 itself is a pacifier teat, i.e. not adapted to dispense nutrition. The base section 27 of the teat assembly 19 has the same outer diameter as the backing section 18 and rests on the upper surface 23 of the backing section, with the teat 26 of the teat assembly 19 projecting away from the backing section 18.

To provide an infant (not shown) with appropriate olfactory cues while it is pacified, the odor carrier of the pacifier, which takes the shape of an annular cotton pad 20 as described in the first embodiment, is infused with a female's odor, for example by being brought into contact with skin, and then placed on the base section 27 of the teat assembly 19. The annular cotton pad 20 has the same outer diameter as the base section 27 of the teat assembly 19 and a central hole 28 that accommodates the teat 26, and therefore rests horizontally on the base section 27, surrounding the teat 26 at its base. Once placed onto the base section 27 of the teat assembly 19 the pad 20 is held in place by the cap 21, which has the same structure as the container cap 4 described in the first embodiment and is screwed onto the backing section 18 to hold the individual components of the pacifier 17 together. Again, an annular cover 22 comprising a central opening 22a for accommodating the teat 26 is provided to allow the diffusion openings 29 to be closed when required.

In use, odor diffuses from the pad 20, through the diffusion openings 29 in the cap 21 into the surroundings of the pacifier 17, thereby providing olfactory cues for an infant suckling the teat 26.

A number of alternative odor carriers are suitable for both the first and second embodiment and will now be described. The features of each of these odor carriers may be embodied individually or in any combination.

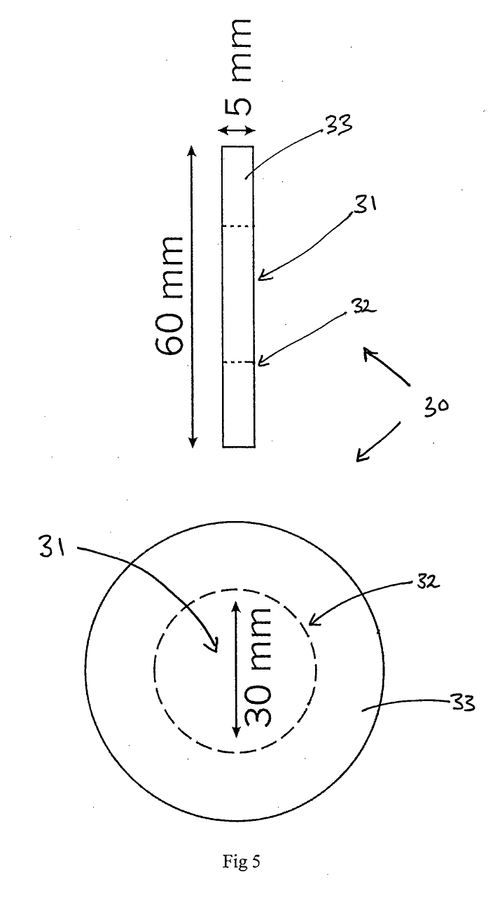

With reference to FIG. 5, a first alternative odor carrier 30 has the same structure as the pad described as part of the first and second embodiments of the invention, with the exception that the central opening is filled with a circular removable section 31. A perforated border 32 separates the removable section from the annular remainder 33 of the pad.

The first alternative odor carrier 30 is particularly suitable for absorbing breast odor from lactating females. It is designed to be held in place, in its entirety, including the removable section 31, on a female's breast (not shown) in the manner of a nursing pad: the removable section 31 is aligned with the nipple of the breast. Whilst in contact with the breast, the odor carrier 30 as a whole absorbs odor, and leaked breast milk accumulates predominantly in the circular removable section 31. Since breast milk is prone to relatively rapid decay leading to a build-up of bacteria and malodor, the removable section 31 is separated with the help of the perforated border 31 and discarded after odor absorption is complete, so that the remainder 33 of the odor carrier 30 can be used for example in a feeding device 1 or pacifier 17 according to the first or second embodiment.

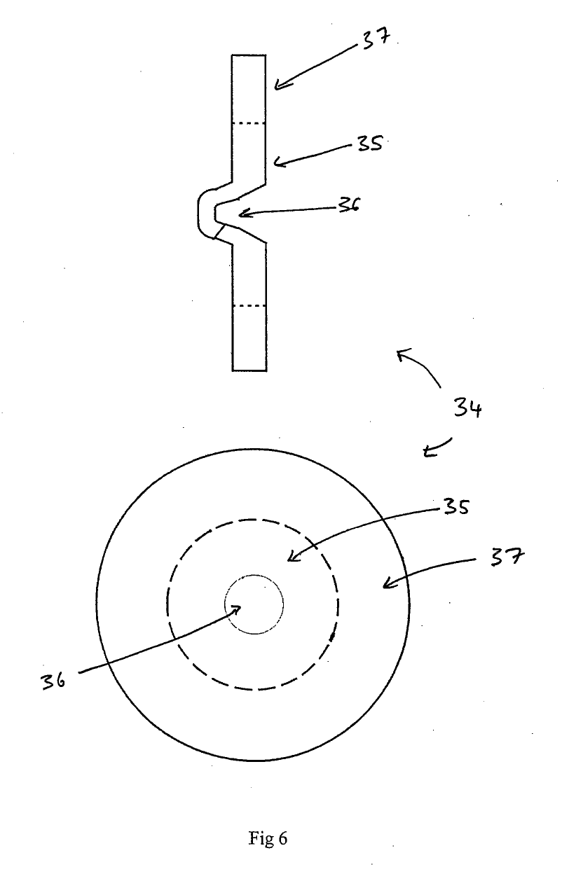

FIG. 6 shows a second alternative odor carrier 34, also particularly suitable for absorbing lactating female breast odor, having a structure similar to that of the first alternative odor carrier 30, with the exception that its removable section 35 comprises an indent 36 at its centre. The second alternative odor carrier 34 is applied to a breast in the same way as the first alternative odor carrier 30 and its indent 36 is shaped so as to accommodate the nipple of the breast, making the second alternative odor carrier 34 easier and more comfortable to wear during odor absorption and/or use of the odor carrier 34 as a nursing pad. Once absorption is complete, the removable section 35 is discarded to leave an annular remainder 37 suitable for use in the first or second embodiment described above and with a comparatively lower concentration of infused breast milk.

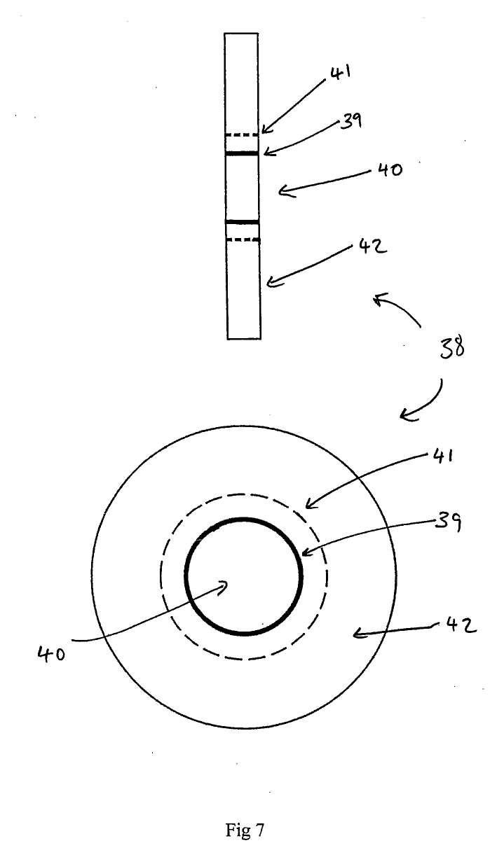

With reference to FIG. 7, a third alternative odor carrier 38 particularly suitable for absorbing lactating female breast odor, is applied in the same way as, and comprises a similar structure to, the first alternative odor carrier 30, but additionally includes a liquid impermeable circular barrier 39 within the circular removable section 40 to prevent breast milk from penetrating outwards from the centre of the odor carrier 38. The circular barrier 39 is concentric with the circular removable section 40 but has a marginally smaller diameter, thereby creating a stop for breast milk just within the circular perforated border 41. Once absorption is complete, the removable section 40 is discarded to leave an annular remainder 42 suitable for use in the first or second embodiment described above and with a comparatively lower concentration of infused breast milk.

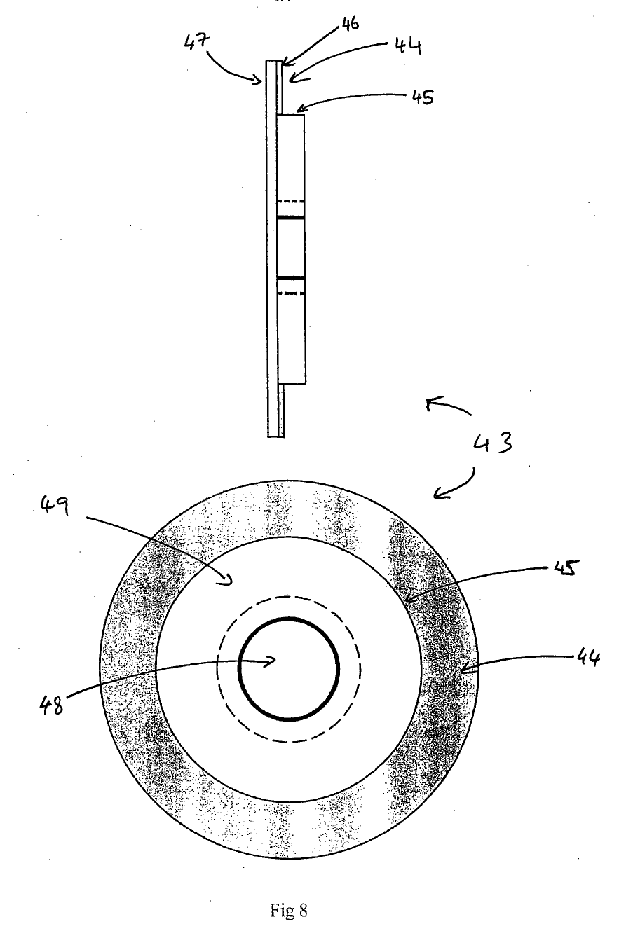

With reference to FIG. 8, a fourth alternative odor carrier 43 particularly suitable for absorbing lactating female breast odor comprises all the features of the third alternative odor carrier 38 and additionally includes a removable adhesive portion 44 suitable for affixing the odor carrier 43 to human skin and/or clothing. The adhesive portion 43 extends at a fixed depth from the outer perimeter 45 of the pad of the odor carrier 43 and comprises a layer of adhesive 46 on one side. A suitably perforated backing sheet 47, which does not prevent removal of the circular removable section 48 is used to removably combine the adhesive portion 44 and the pad. Once absorption is complete, the odor carrier 43 is detached from a breast and/or clothing and the removable and adhesive sections 48, 44 are discarded. This leaves an annular remainder 49 suitable for use in the first or second embodiment described above and with a comparatively low concentration of infused breast milk. In an alternative arrangement (not shown) adhesive is applied directly to the pad, dispensing with the need of a distinct adhesive portion.

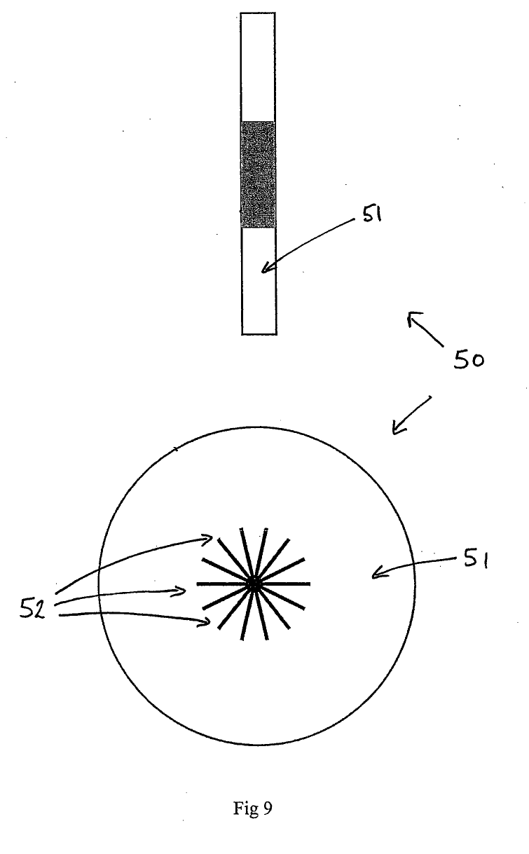

With reference to FIG. 9, a fifth alternative odor carrier 50 comprises a disk shaped cotton pad 51 having a plurality of slits 52 extending radially from its centre. The slits 52 extend up to a circular perimeter centred in the centre of the pad 51 and having a diameter equivalent to that of the central opening 16; 28 in the odor carrier 5; 20 described as part of the first and second embodiments. They represent an alternative way of adapting an odor carrier to allow it to co-operate with the devices of the first and second embodiment.

A sixth alternative odor carrier (not shown) comprises a disk shaped cotton pad having front and rear planar faces and an annular cotton section affixed concentrically and removably to the rear planar face of the cotton pad. When brought into contact with a female's breast rear face first the cotton pad acts as a nursing pad, while the annular cotton section surrounds the nipple of the breast and absorbs odor. Once removed from the cotton pad the annular cotton section may be used, for example as an odor carrier in the devices of the first or second embodiment.

The embodiments described above are purely exemplary: the addition of an odor to a feeding device or a pacifier can be achieved in a variety of ways, all of which are encompassed and compatible with the present invention. The basic structure of the feeding device, the pacifier and their individual components may vary. For instance, a range of shapes such as circles, squared or triangles, or even pores of any size may be used as diffusion openings. Similarly the feeding device itself does not have to take the shape of a bottle and may, for instance, also be a system of gastronasal tubes.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method of feeding an infant, the method comprising the steps of:
providing a feeding device;
adding a female's breast odor to the feeding device; and
feeding the infant using the feeding device.

2. The method of claim 1, wherein the female's breast odor is produced naturally.

3. The method of claim 1, wherein the female's breast odor is that of a lactating female.

4. The method of claim 1, wherein the female's breast odor is that of the infant's mother.

5. The method of claim 4, wherein the mother's breast odor is that of the infant's biological mother.

6. The method of claim 1, wherein the step of adding the female's breast odor to the feeding device comprises the sub-steps of:
providing an odor carrier;
bringing the odor carrier into contact with a female's breast to allow the breast odor to be absorbed by the odor carrier; and
combining the odor carrier with the feeding device.

7. The method of claim 6, further comprising the sub-step of treating the odor carrier with a solvent and/or emollient, prior to bringing the odor carrier into contact with the female's breast.

8. The method of claim 6, further comprising the sub-step of using the odor carrier as a nursing pad for absorbing leaked breast milk, prior to combining the odor carrier with the feeding device.

9. The method of claim 8, further comprising the sub-step of discarding a section of the odor carrier, after using the odor carrier as a nursing pad but prior to combining the odor carrier with the feeding device, and wherein the discarded section of the odor carrier is selected to contain a higher concentration of leaked breast milk than the remainder of the odor carrier.

10. A feeding device for feeding an infant, the device including nutrition storage means and a nutrition dispenser for dispensing stored nutrition, wherein the device is adapted to co-operate with an odor carrier carrying a female's breast odor such that the infant can be exposed to the female breast odor during feeding.

11. A feeding device according to claim 10, comprising an odor carrier carrying a female's breast odor for exposing the infant to the female breast odor during feeding.

12. A feeding device according to claim 11, wherein the female breast odor is produced naturally.

13. A feeding device according to claim 11, wherein the female breast odor is that of a lactating female.

14. A feeding device according to claim 11, wherein the female breast odor is that of the infant's mother.

15. A feeding device according to claim 14, wherein the female breast odor is that of the infant's biological mother.

16. A feeding device according to claim 11, wherein the odor carrier is located in the vicinity of the nutrition dispenser.

17. A feeding device according to claim 11, wherein the nutrition dispenser comprises a teat and the odor carrier is adapted to co-operate with the teat.

18. A feeding device according to claim 11, wherein the odor carrier is housed below at least one outer surface of the feeding device and said at least one outer surface comprises at least one diffusion opening for allowing odor to diffuse from the odor carrier and out of the feeding device.

19. A feeding device according to claim 18, further comprising means for selectively closing at least one diffusion opening.

20. A pacifying device for pacifying an infant, characterised in that it is adapted to co-operate with an odor carrier carrying a female's odor such that the infant can be exposed to the female's odor when using the pacifier.

21. A pacifying device according to claim 20, further comprising an odor carrier carrying a female's odor.

22. A pacifying device according to claim 21, wherein the female odor is produced naturally.

23. A pacifying device according to claim 21, wherein the female odor is axillary odor.

24. A pacifying device according to claim 21, wherein the female odor comprises neck odor.

25. A pacifying device according to claim 21, wherein the female odor is that of the infant's mother.

26. A pacifying device according to claim 25, wherein the female odor is that of the infant's biological mother.

27. A pacifying device according to claims 21, comprising a teat and wherein the odor carrier is adapted to co-operate with the teat.

28. A pacifying device according to claim 21, wherein the odor carrier is housed below at least one outer surface of the pacifying device and said at least one outer surface comprises at least one diffusion opening for allowing odor to diffuse from the odor carrier and out of the pacifying device.

29. A pacifying device according to claim 28 further comprising means for selectively closing at least one diffusion opening.

30. An odor carrier comprising an absorbent portion for absorbing odor, and being adapted to co-operate with a feeding or pacifying device such that an infant is exposed to odor absorbed by the odor carrier during use of said feeding or pacifying device.

31. An odor carrier according to claim 30 adapted to be usable as a nursing pad.

32. An odor carrier according to claim 30, wherein the absorbent portion comprises a pad in the shape of a disk.

33. An odor carrier according to claim 32, wherein the pad comprises a removable section.

34. An odor carrier according to claim 33, wherein the removable section is disk shaped and concentric with the disk shaped pad.

35. An odor carrier according to claim 33, wherein the removable section is separated from the remainder of the disk shaped pad by a perforated border.

36. An odor carrier according to claim 33, comprising an indent for accommodating the nipple of a breast, the indent being located on the removable section.

37. An odor carrier according to claim 33, comprising a liquid impermeable barrier for preventing the spread of liquid to a part of the pad, the barrier being located in the removable section and being suitable for preventing the spread of liquid out of the removable section.

38. An odor carrier according to claim 32, wherein the pad comprises at least one slit to allow the odor carrier to co-operate with a feeding or pacifying device.

39. An odor carrier according to claim 30, adapted to be held below at least one outer surface of a feeding or pacifying device.
